(19) United States
(12) Patent Application Publication
(10) Pub. No.: US 2025/0269467 A1
(43) Pub. Date: Aug. 28, 2025

(54) LASER WELDING OF OPTICAL FIBERS

(71) Applicant: Alcon Inc., Fribourg (CH)

(72) Inventors: Kevin Canzone, Coto de Caza, CA (US); Gisele Maxwell, Temecula, CA (US)

(21) Appl. No.: 19/027,114

(22) Filed: Jan. 17, 2025

Related U.S. Application Data
(60) Provisional application No. 63/558,702, filed on Feb. 28, 2024.

(51) Int. Cl. B23K 26/20; B23K 26/70; G02B 6/46

(52) U.S. Cl. CPC B23K 26/20; B23K 26/703; G02B 6/46

(57) ABSTRACT

Certain embodiments disclosed herein provide systems and methods for coupling an optical fiber to a ferrule. For example, certain aspects provide a method for coupling an optical fiber to a ferrule including inserting the optical fiber through a ferrule such that an end of the optical fiber protrudes beyond an end of the ferrule and applying a laser beam to the end of the optical fiber, wherein the laser beam melts a portion of the optical fiber. The method further includes securing, through cooling of the melted portion of the optical fiber, the optical fiber to the ferrule, wherein the laser beam melts a portion of the optical fiber to create a fluid which seals the optical fiber to the ferrule upon solidifying.

In a wide variety of medical procedures, laser light (e.g., a laser treatment beam (“treatment beam”), or laser aiming beam (“aiming beam”), etc.) is used to assist in surgery and/or treat patient anatomy. For example, in laser photocoagulation, a laser probe propagates a laser treatment beam to cauterize blood vessels at a burn spot across the retina. In another example, a laser probe may be used to cut vitreous in a vitrectomy procedure. In such examples, a laser beam is typically transmitted from a surgical laser system through an optical fiber that proximally terminates in a port connector, which connects to the surgical laser system, and distally terminates in a laser probe, which is manipulated by a surgeon.

In order to effectively and reliably transmit laser light, an optical fiber having secure connection(s) with the surgical console through the port connector, and/or to neighboring laser fibers (e.g., within the laser probe), is required. Existing methods of coupling optical fibers to other components include bonding optical fibers to ferrules that may be positioned in contact with other ferrules or connectors at optical junctions. Typically, the optical fibers are bonded to ferrules with adhesives, which are prone to failure, do not effectively withstand heat (e.g., when the probe is autoclaved), and may affect the transmission of optical energy through the optical fibers.

### SUMMARY

Aspects of the present disclosure relate to a method of laser welding for use in surgical settings, and more specifically, laser welding configured to weld optical fibers to alignment ferrules.

Certain embodiments of the present disclosure provide an optical fiber system including an optical fiber having a proximal end and a ferrule for coupling the proximal end of the optical fiber to a surgical laser system. The optical fiber is fused to the ferrule using a laser-based fusion process.

Certain embodiments of the present disclosure provide a method for coupling an optical fiber to a ferrule including inserting the optical fiber through a ferrule such that an end of the optical fiber protrudes beyond the end of the ferrule. The method further includes applying a laser beam to the end of the optical fiber, wherein the laser beam melts a portion of the optical fiber and securing, through the cooling of the melted portion of the optical fiber, the optical fiber to the ferrule, wherein the laser beam melts a portion of the optical fiber to create a fluid which seals the optical fiber to the ferrule upon solidifying.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a schematic side view of a system for generating laser beams for delivery to a surgical target, according to embodiments described herein.

FIGS. 2A-2B illustrate cross-sectional side views of an exemplary optical port that may be implemented in the surgical system of FIG. 1A, according to embodiments described herein.

FIG. 3A illustrates a longitudinal cross-sectional view of an exemplary surgical hand piece device that may be implemented in the surgical system of FIG. 1A, according to embodiments described herein.

FIG. 3B illustrates a longitudinal cross-sectional view of the coupling of a first optical fiber and a second optical fiber at a coupling interface, according to embodiments described herein.

FIG. 4A is a flow diagram of an example method for coupling optical fiber to a ferrule via laser welding, according to embodiments described herein.

FIGS. 4B-4F illustrate various schematics corresponding to the method of FIG. 4A, according to embodiments described herein.

FIG. 5 illustrates a cross-sectional view of a ferrule, according to certain embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or operations described with respect to one implementation may be combined with the features, components, and/or operations described with respect to other implementations of the present disclosure.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical laser system.

Embodiments disclosed herein generally relate to systems and methods for attaching optical fibers to alignment ferrules for use with surgical systems, such as methods of welding optical fibers to alignment ferrules at optical fiber coupling interfaces. The systems and methods described herein may be utilized in combination with any suitable surgical instruments having laser light emitting functionality, such as those described herein. For example, the systems and methods described herein may be utilized in combination with ophthalmic surgical instruments.

Certain embodiments of the present disclosure include systems and techniques for bonding an optical fiber to a ferrule at a coupling interface of the optical fiber via laser welding. Such welding of the optical fiber to the ferrule provides improved transmission of optical energy to and/or from the optical fiber and overcomes many of the issues associated with certain existing methods of coupling optical fibers to corresponding ferrules.

For example, certain existing methods include bonding optical fibers to ferrules via adhesive bonding substrates. However, such adhesives are prone to failure, which can cause complete failure at an optical junction or complete failure of a surgical instrument. Further, adhesives are typically not resistant to high temperatures, which can cause failure in applications requiring high temperatures (e.g., optical fibers requiring autoclaving between uses). Still further, adhesives can be affected by exposure to chemicals, and may generally affect the transmission of optical energy to and/or from an optical fiber.

Other existing methods of bonding optical fibers to ferrules include soft soldering. However, soft soldering techniques typically involve manual, tedious, and repetitive processes that utilize multiple coatings of various materials that can affect the transmission of optical energy to and/or from an optical fiber.

Accordingly, the laser welding techniques described herein provide reliable and efficient coupling of optical fibers to ferrules while mitigating at least the above constraints related to existing bonding methods. For example, the laser welding techniques described herein facilitate ferrule-optical fiber bonds with improved chemical resistance, temperature resistance, and mechanical resistance, without affecting optical performance. Further, the laser welding techniques described herein decrease the potential for user error related to prepping the surfaces of the ferrule or the optical fiber to bind to the adhesive, or the preparation of the adhesive itself. Such laser-welded ferrules/optical fibers may be used at coupling interfaces to ensure the coupling interfaces are secure and reliable without affecting optical performance. In certain embodiments, the coupling interfaces may be between adjacent optical fibers (e.g., butt couplings), etc. In other embodiments, the coupling interfaces may be between the optical fiber and an optical component of a surgical laser system, such as an output optical port of a laser source, etc.

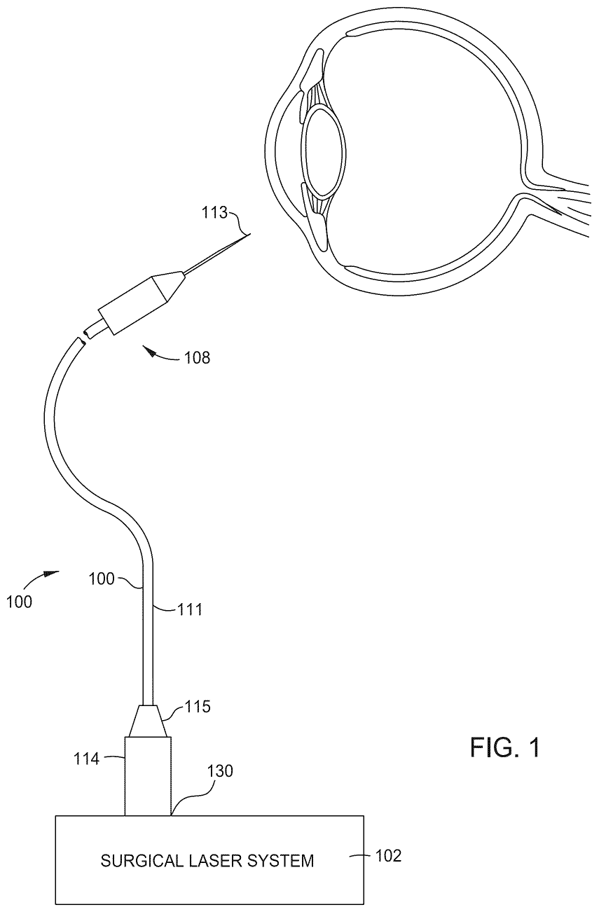

FIG. 1 illustrates an example surgical system 100 for performing a laser-assisted ophthalmic surgical procedure. Although illustrated as an ophthalmic surgical procedure, note that the systems and methods described herein may be utilized in combination with, or for, any suitable surgical instruments, or other instruments, having laser light emitting functionality.

Surgical system 100 includes a laser system 102, having one or more laser and/or illumination sources for generating laser and/or illumination beams 113. Laser and/or illumination beam(s) 113 are representative of one or more types of laser aiming beams, laser treatment beams, and/or illumination light beams. In one example, the laser and/or illumination beam(s) 113 include laser light having a wavelength of about 1 μm (micrometer) to about 10 μm, such as about 3 μm and may be suitable for, for example, photoemulsification, laser vitrectomy, or other types of tissue removal. In another example, laser and/or illumination beams 113 include laser light having a wavelength appropriate for photocoagulation to treat retinal tears and detachments, etc. The user, such as a surgeon, may toggle the laser between on positions and off positions using a switch on a probe 108, a foot pedal, or other means.

The probe 108 is meant to be representative of a probe that may be used in a variety of ophthalmic surgical systems, such as for photocoagulation in retinal surgery, photoemulsification in cataract surgery, vitrectomy, or other ophthalmic surgical procedures. For example, the surgeon may activate a laser beam, such as through a foot pedal or other means, to emulsify and remove the patient's cataract, cut and remove the patient's vitreous, perform photocoagulation, or the like. In further examples the laser system 102 includes an illumination system including one or more illumination sources.

In certain embodiments, surgical laser system 102 is integrated with one or more additional devices and/or systems. For example, in certain embodiments, surgical laser system 102 may be integrated in a surgical console, such as a surgical console for performing ophthalmic surgical procedures, including vitreoretinal procedures, cataract surgeries, corneal transplants, glaucoma surgeries, LASIK (laser assisted in situ keratomileusis) surgeries, refractive lens exchanges, trabeculectomies, keratotomy procedures, and/or keratoplasty surgeries, etc. Consoles that are capable of performing two or more of these procedures are also within the scope of this disclosure. An example of a console configured for performing vitreoretinal procedures is the Constellation® System available from Alcon Laboratories, Inc., Fort Worth, Texas. An example of a console configured for performing cataract surgeries is the Centurion® System available from Alcon Laboratories, Inc., Fort Worth, Texas.

The surgical system 100 includes a connector 114, an optical fiber system including an optical fiber, an optical fiber cable 111, and the probe 108. The optical fiber may be at least partially housed inside the optical fiber cable 111. A distal end of the optical fiber cable 111 couples to the probe 108 and a proximal end of the optical fiber cable 111 couples to a connector 114. In some cases, the optical fiber may include more than one fiber. The optical fiber may be a single-crystal sapphire optical fiber made from α-Al2O3. In other embodiments, the single-crystal optical fiber of the optical fiber may be made of Ti:Sapphire, Y3Al5O12 (YAG), Ho:YAG, Yb:YAG, Nd:YAG, Er:YAG, Ce:YAG, Cr:YAG, or ZrF4—BaF2—LaF3—AlF3—NaF (ZBLAN). However, any material capable of propagating a laser light and wavelength thereof may be used and are also contemplated.

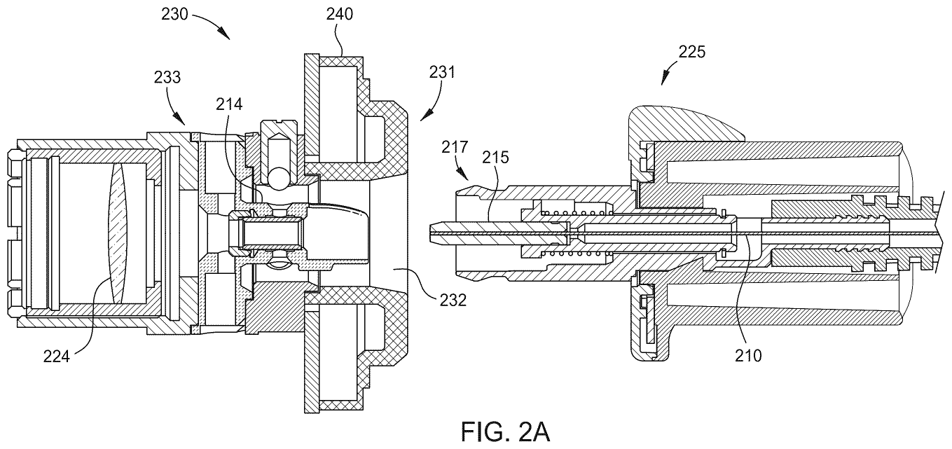

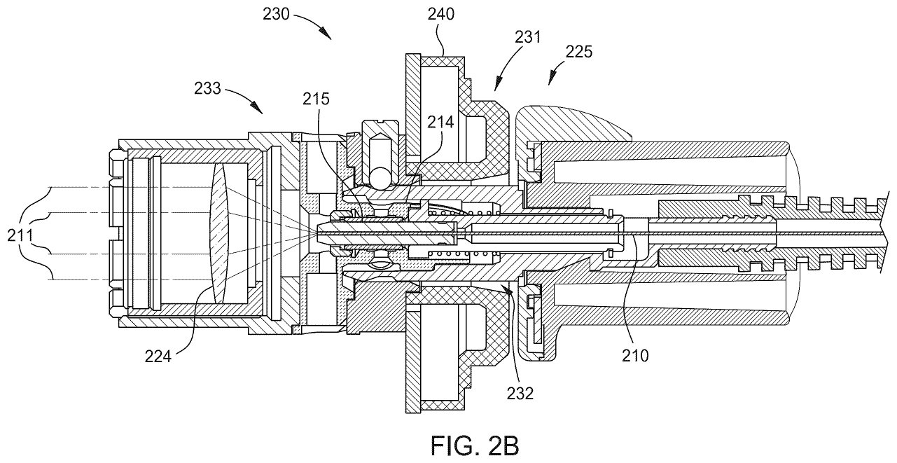

The connector 114 couples to an optical port (e.g., optical port 230 as described in reference to FIGS. 2A-2B) of laser system 102. The optical fiber extends through the connector 114 toward the optical port. The connector 114 may include a ferrule 115 with an opening into which a proximal end of optical fiberand a proximal end of the optical fiber cable 111 are inserted. The proximal end of optical fiber includes an interface upon which laser beams from surgical laser system 102 may be focused when the proximal end of optical fiber is inserted into the ferrule 115. The interface of the optical fiber comprises the exposed proximal ends of the one or more cores to which laser beams may be directed.

FIGS. 2A-2B illustrate cross-sectional side views of an example optical port 230 of a surgical laser system, including an example port adapter, and an example optical fiber connector in an unmated state and a mated state, respectively, in accordance with certain embodiments of the present disclosure. The embodiments described herein are provided for illustrative purposes, as an example of the positioning of the ferrule and optical fiber (e.g., at the port of the laser system). In certain embodiments, optical port 230 is representative of optical port 130 in FIG. 1.

As shown, optical port 230 includes port adapter 214, which is at least partially disposed through an opening 232 in a cover plate 240 at distal end 231 of optical port 230. In certain embodiments, cover plate 240 couples to an outer panel of a surgical laser system and/or surgical console for performing ophthalmic surgical procedures, such as a vitreoretinal surgical console or a phacoemulsification console. An opposing, proximal end 233 of optical port 230 is disposed adjacent to a condensing lens (or focusing lens) 224, which is configured to focus illumination and/or laser beams onto optical fiber 210 disposed within optical fiber connector 225. Together, optical port 230 and condensing lens 224 may be referred to as a “chimney” or a “high power connector.” Similar to optical fiber described above, optical fiber 210 may be a single-core optical fiber (SCF) or a multi-core optical fiber (MCF).

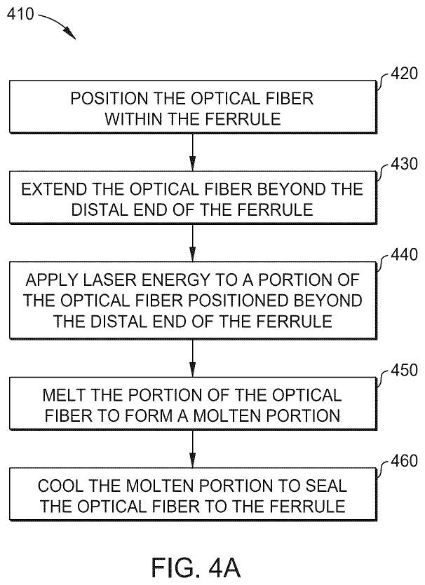

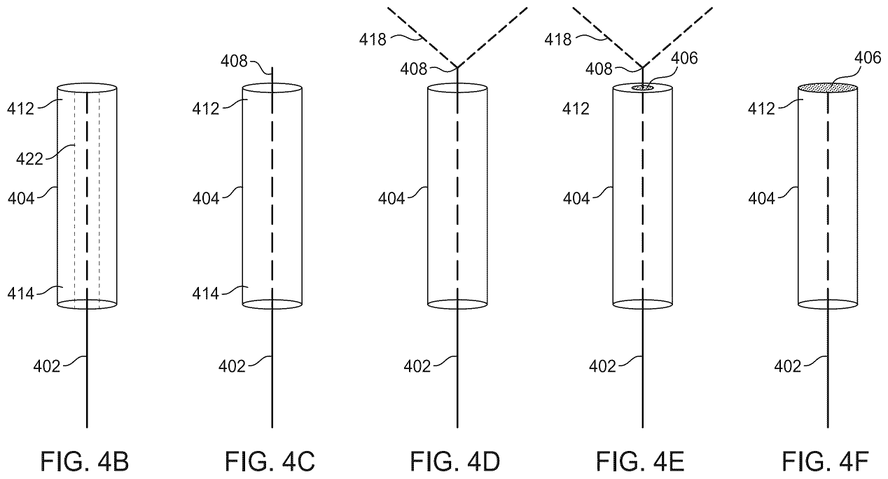

A proximal end of the optical fiber 210 is bonded to the proximal end 217 of a ferrule 215, which is disposed within a proximal end of optical fiber connector 225. In some embodiments, ferrule 215 may be, or include, a metallic tube, a ceramic tube, a sapphire tube, a single crystal tube, or other material. Ferrule 215 and optical fiber 210 are angularly aligned and angularly fixed within optical fiber connector 225. The ferrule 215 may be coupled and/or sealed to the optical fiber 210 through the laser welding process as described in reference to FIG. 4.

As shown in FIG. 2B, when fully inserted into optical port 230, a proximal end of ferrule 215 is disposed within port adapter 214 such that condensing lens 224 may converge illumination, aiming, and/or treatment beams 211 into an interface plane of the proximal end of optical fiber 210. During insertion, port adapter 214 and optical fiber connector 225 mechanically guide the position and angular orientation of optical fiber connector 225 and ferrule 215 such that each core of optical fiber 210 is aligned with one or more illumination, aiming, and/or treatment beams 211 in a final, “connected” position. As described herein, laser welding of the ferrule 215 to the optical fiber 210 may provide improved chemical, mechanical, and temperature resistance of the optical fiber 210 and the ferrule 215 at the optical port 230. Laser welding may provide the improved chemical, mechanical, and temperature resistance while maintaining optical transmission from the laser system 102 into the optical fiber 210.

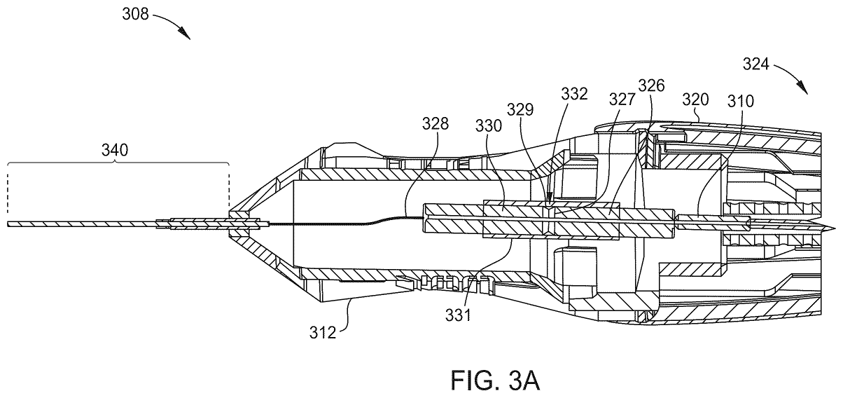

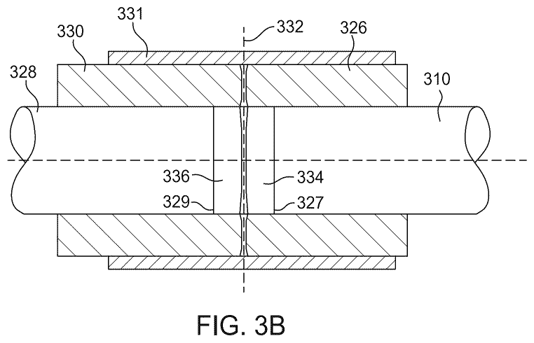

FIGS. 3A-3B illustrate cross-sectional side views of an example probe 308 of a surgical laser system, and an example interface between optical fibers in the probe, in accordance with certain embodiments of the present disclosure. In certain embodiments, probe 308 is representative of probe 108 in FIG. 1. FIGS. 3A-3B are provided for illustrative purposes, as an example of the positioning of the ferrule and the optical fiber (e.g., at a butt coupling between fibers within a surgical probe).

As shown, within surgical hand piece 312 of probe 308, a first optical fiber 310 is in optical contact with a second optical fiber 328. In this example, the first optical fiber 310 optically couples the second optical fiber 328, and thus the probe 308, to an optical port of a surgical laser system, and propagates laser and/or illumination light beams from the surgical laser system to the second optical fiber 328. A proximal end of the first optical fiber 310 connects to the optical port of the surgical laser system, while a distal end 327 of the first optical fiber 310 extends into a housing 320 of hand piece 312 from a proximal end 324 of the housing 320. The first optical fiber 310 may be similar to optical fiber 210 of FIGS. 2A-2B where the optical fiber 210 extends from the surgical laser system to the probe. The distal end 327 of the first optical fiber 310 is optically and mechanically coupled to a proximal end 329 of the second optical fiber 328 at a coupling interface 332. The second optical fiber 328 extends from the coupling interface 332 and through probe tip 340 to a distal end thereof. Accordingly, the second optical fiber 328 transmits laser and/or illumination light beams received from the first optical fiber 310 through the probe 308 and out of the distal end of probe tip 340.

As better seen in FIG. 3B, at the coupling interface 332, the first optical fiber 310 may include window 334 at the distal end 327 and the second optical fiber 328 may include window 336 at the proximal end 329. A ferrule 326 is attached to (e.g., around) the window 334 at the distal end 327 of the first optical fiber 310 and disposed adjacent to a ferrule 330 attached to the window 336 at the proximal end 329 of the second optical fiber 328. As shown, the window 334 at the distal end 327 of the optical fiber 310, and the ferrule 326 disposed thereon, are in optical contact (e.g., butt coupled) with the window 336 at the proximal end 329 of the second optical fiber 328 and the ferrule 330, respectively, such that laser light may be transmitted from the first optical fiber 310 to the second optical fiber 328 through windows 334 and 336. In certain other embodiments, the distal end of the optical fiber 310, and the ferrule 326 disposed thereon, may be in direct optical contact with the proximal end 329 of the second optical fiber 328 and the ferrule 330, such that laser light may be directly transmitted from the first optical fiber 310 to the second optical fiber 328.

The windows 334 and 336 may comprise an optically clear or transparent material. In certain aspects, the transparent material has optical power and, in certain other aspects, the transparent material does not have optical power. Optical power (also referred to as dioptric power, refractive power, focusing power, or convergence power) is the degree to which a lens, mirror, or other optical system converges or diverges light. Accordingly, the windows 334 and 336 may itself be a lens, such as a spherical lens having rounded ends or a non-spherical lens having flat ends. Examples of the transparent material of the windows 334 and 336 include sapphire, fused silica, or other glass or ceramics materials. Although windows 334 and 336 are shown in FIG. 3B in a particular arrangement at the ends of optical fibers 310 and 328, is it also contemplated that any of the optical fibers disclosed herein may have a window fused to a proximal and/or distal end thereof.

The first optical fiber 310 and the second optical fiber 328 may be retained in position near the coupling interface 332 by the ferrules 326 and 330, respectively. In some embodiments, ferrules 326, 330 may be, or include, a metallic tube, a ceramic tube, a sapphire tube, a single crystal tube, or other material. The first optical fiber 310 and second optical fiber 328 may be configured to have a diameter slightly smaller than the ferrules 326 and 330, respectively, such that the first optical fiber 310 and second optical fiber 328 may be slid into the internal channels of the ferrules 326 and 330. The optical fiber 310 and the second optical fiber 328 may be welded with the ferrules 326 and 330, respectively, as described in reference to FIG. 4.

In certain embodiments, a portion of the first optical fiber 310, the second optical fiber 328, and the ferrules 326, 330 at the coupling interface 332 may be further disposed within a sleeve 331 to maintain the optical connection between the first optical fiber 310 and the second optical fiber 328. In some embodiments, the sleeve 331 includes a cylindrical tube configured to clamp down on the coupling ends of the ferrules 326, 330 and the ends of the optical fiber 310 and the second optical fiber 328 to reduce transmission loss at the coupling interface 332.

FIG. 4A-4F illustrate an example method and corresponding schematics for bonding an optical fiber to a ferrule via laser welding, according to embodiments described herein. The method 410 in FIG. 4A and corresponding schematics demonstrating the method in FIG. 4B-4F are described together for clarity.

Method 410 in FIG. 4A may be performed in a different order than shown in FIG. 4A, additional operations may be provided before, during, and/or after the described operations, and/or some of the operations described may be replaced or eliminated in other implementations. While the method 410 described herein is discussed in reference to a ferrule 404, the method 410 may be applied to welding optical fibers and any type of connector housing and/or adapter disposed around the optical fibers. In certain embodiments, the method may also include operations that deposit additional scaling and/or bonding materials to the optical fiber 402 and the ferrule 404 to ensure sufficient material is present to melt and bond the optical fiber to the ferrule.

As described herein and shown in FIG. 4A-4F, the method 410 is described with reference to distal ends of the optical fiber 402 and the ferrule 404. However, the method 410 may be used for securing any end, or other portion, of an optical fiber to any end, or other portion, of a ferrule, including any ends or portions of either the optical fiber or ferrule that are to be positioned at a coupling interface with, e.g., another ferrule/optical fiber or optical port, as described in FIGS. 2A and 2B.

Turning now to FIG. 4A, the method 410 may begin at block 420 by positioning the optical fiber 402 within an internal channel 422 of the ferrule 404, as shown in FIG. 4B. The internal channel 422 of the ferrule 404 may be configured to have a diameter that is slightly larger than the optical fiber 402. In certain embodiments, the optical fiber 402 may be inserted through the proximal end 414 of the ferrule 404, through the length of the internal channel 422 of the ferrule 404 to the distal end 412 of the ferrule 404.

At block 430, the optical fiber 402 is inserted through the ferrule 404 such that a portion 408 of the optical fiber 402 extends beyond the distal end 412 of the ferrule 404. As shown in FIG. 4C, the optical fiber 402 may be inserted through the ferrule 404 until the portion 408 of the optical fiber 402 is extended beyond the ferrule 404.

At block 440, once the portion 408 of the optical fiber 402 is extended slightly beyond the ferrule 404, a laser energy 418 may be applied to the portion 408 of the optical fiber 402 to melt the portion 408 of the optical fiber 402, as shown in FIG. 4D. In certain embodiments, the laser energy may be provided by a carbon dioxide (CO₂) laser source. In certain other embodiments, the laser energy may be provided by a three micron laser source, etc. The laser energy may have a power output range of between 0 to 60 watts, such as between 10 to 50 watts, 20 to 40 watts, 0 to 20 watts, or 0 to 30 watts. Though the laser energy power output is described as being between 0 to 60 watts, laser energy power outputs outside of this range are also contemplated.

The laser energy may be delivered as a single laser beam, such as a donut-shaped laser beam focused on a single point using parabolic mirrors, or a filled laser beam focused with a lens. However, other shapes of laser beams are also contemplated. The laser energy may be emitted in pulses having a desired duration and repetition rate. In certain embodiments, the laser energy may be emitted in a continuous manner. The laser energy may be applied to a single location of the optical fiber, such as the portion of the optical fiber that meets the end of the ferrule, or the laser energy may be rotated such that the laser energy is provided to various locations around or along the optical fiber.

At block 450, the laser energy 418 may melt the portion 408 of the optical fiber 402 to form a molten portion 406, as shown in FIG. 4E. The molten portion 406 may be formed of the entirety of the portion 408 of the optical fiber 402 that extends beyond the ferrule 404. In certain other embodiments, the molten portion 406 is formed of a segment of the portion 408 of the optical fiber 402 that extends beyond the ferrule 404.

The molten portion 406 may form an end of the optical fiber 402 with a larger diameter than the rest of the optical fiber 402 upon solidifying. For example, when the portion 408 of the optical fiber 402 is melted, the molten portion 406 may settle within the entire diameter of the internal channel of the ferrule 404.

At block 460, the molten portion 406 is cooled to securely seal the optical fiber 402 to the ferrule 404 when the molten portion 406 has solidified, as shown in FIG. 4F.

In certain embodiments, upon solidifying, the molten portion 406 may extend beyond the end of the ferrule 404. To properly align the end of the optical fiber 402 with the distal end 412 of the ferrule 404, the solidified molten portion 406 may be polished. In certain embodiments, the solidified molten portion 406 may be polished using chemical mechanical planarization (CMP). Following securing the optical fiber 402 to the ferrule 404 according to the method 410 described herein, the optical fiber 402 and ferrule 404 may be used to optically couple an optical fiber to an optical port of a surgical laser system as described with reference to FIGS. 2A and 2B, or to optically couple a first and second optical fiber within a hand piece as described with reference to FIGS. 3A and 3B.

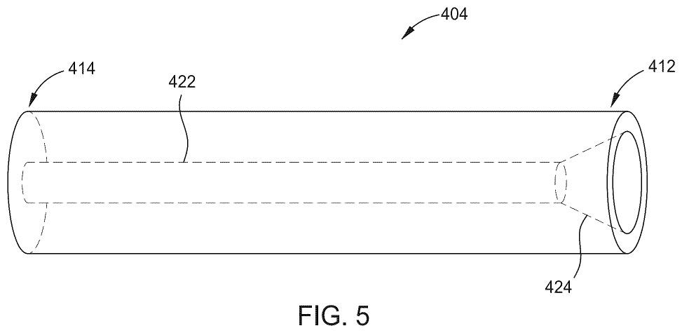

In certain embodiments, an end of the ferrule 404, shown as the distal end 412 of the ferrule 404 in FIG. 5, may comprise a cone-shaped portion 424 of the internal channel 422, which is configured to have the larger diameter end of the cone-shape at the distal-most end of the ferrule 404. Further, the smaller diameter end of the cone-shape portion 424 may be coupled to the existing internal channel of the ferrule 404 some distance toward the proximal end 414 of the ferrule 404. The cone-shaped portion 424 of the internal channel 422 is configured to allow the molten portion 406 to occupy the entire volume of the cone-shaped portion 424 of the internal channel 422. The cone-shaped portion 424 may be utilized to increase the thermal capacity of the optical fiber 402 and/or increase the damage threshold of the optical fiber 402. While the distal end 412 of the ferrule 404 is described with reference to FIG. 5, the cone-shaped portion 424 may be present at any end, or other portion, of a ferrule, including any ends or portions of the ferrule that are to be positioned at a coupling interface with, e.g., another ferrule/fiber or optical port, as described in FIGS. 2A and 2B.

In certain embodiments, rather than directly delivering laser energy to the optical fiber to melt the optical fiber, an intermediate material layer is applied over the optical fiber and melted using the laser energy. In other words, the intermediate material forms the molten portion 406 used to weld the optical fiber to the ferrule. For example, a user may manually place the intermediate material within the distal end 412 of the ferrule 404. The laser energy 418 may be applied to the intermediate material to create the molten portion 406 to couple the optical fiber 402 to the ferrule 404. In certain embodiments, the intermediate material may be a donut-shaped material or a window configured to be positioned over the fiber to act as an additional material to create the molten portion 406. The additional material may be configured to fill the larger diameter, and therefore, volume, of the cone-shaped portion 424 of ferrule 404. The intermediate material may be formed of the same material as the optical fiber 402 or a different material, such as a material comprising α-Al2O3 (Sapphire), Ti:Sapphire, Y3Al5O12 (YAG), Ho:YAG, Yb:YAG, Nd:YAG, Er:YAG, Ce:YAG, Cr:YAG, or ZrF4—BaF2—LaF3—AlF3—NaF (ZBLAN). The intermediate material may be a paste, semi-solid, or solid material.

In certain embodiments, one or more of the optical fibers described herein may comprise a window laser welded to one or more ends of the optical fiber, as described with reference to FIG. 3B. In such embodiments, once the window is laser welded to the fiber as described in reference to FIG. 4, the optical fiber-window component may be placed within the internal channel of the ferrule. The window may be utilized in any of the foregoing embodiments. In certain embodiments, the fibers may be attached and/or adjacent to one another without a window laser welded to one or more ends of the optical fibers. In other embodiments, as described herein, the optical fibers may include a window fused to the optical fibers at connection points to other optical fibers as described in FIGS. 3A-3B, or optical fibers at connection points with an optical port as described in FIGS. 2A-2B.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. An optical fiber system, comprising:
an optical fiber having a proximal end and a distal end; and
a ferrule for coupling the proximal end of the optical fiber to a surgical laser system,
wherein the optical fiber is fused to the ferrule using a laser-based fusion process.

2. The optical fiber system of claim 1, wherein the laser-based fusion process comprises:
melting a proximal end of the optical fiber to form a molten portion of the optical fiber; and
allowing the molten portion to solidify and bond with the ferrule.

3. The optical fiber system of claim 2, wherein system further comprises:
a second ferrule for coupling a distal end of the optical fiber to a second optical fiber within a surgical hand piece, wherein the optical fiber is fused to the second ferrule using a laser-based fusion process.

4. The optical fiber system of claim 1, wherein the optical fiber comprises a single-crystal optical fiber composed of a material comprising α-Al2O3 (Sapphire), Ti:Sapphire, Y3Al5P12 (YAG), Ho:YAG, Yb:YAG, Nd:YAG, Er:YAG, Ce:YAG, Cr:YAG, or ZrF4—BaF2—LaF3—AlF3—NaF (ZBLAN).

5. The optical fiber system of claim 1, wherein the ferrule comprises a material from at least ceramic, ZrO2, α-Al2O3 (Sapphire), other single crystal, or metal.

6. The optical fiber system of claim 2, wherein the molten portion of the optical fiber creates a seal between the optical fiber and the ferrule.

7. A method for coupling an optical fiber to a ferrule, comprising:
inserting the optical fiber through a ferrule such that an end of the optical fiber protrudes beyond an end of the ferrule;
applying a laser beam to the end of the optical fiber, wherein the laser beam melts a portion of the optical fiber; and
securing, through cooling of the melted portion of the optical fiber, the optical fiber to the ferrule, wherein the laser beam melts a portion of the optical fiber to create a fluid which seals the optical fiber to the ferrule upon solidifying.

8. The method of claim 7, wherein the method further comprises polishing, using a chemical mechanical planarization method, the end of the optical fiber and the ferrule.

9. The method of claim 7, wherein the melted portion of the optical fiber creates a seal between the optical fiber and the ferrule.

10. The method of claim 7, wherein the ferrule comprises a cone-shaped portion coupled to an internal channel of the ferrule and the cone-shaped portion is occupied by the melted portion of the optical fiber.

11. A method for coupling an optical fiber to a ferrule, comprising:
inserting the optical fiber through the ferrule;
applying a material to an end of the optical fiber within the ferrule;
applying a laser beam to the material at the end of the optical fiber, wherein the laser beam melts the material; and
securing, through cooling of the melted material, the optical fiber to the ferrule, wherein the laser beam melts the material in contact with the optical fiber to create a fluid which couples the optical fiber to the ferrule upon solidifying.

12. The method of claim 11, wherein the method further comprises polishing, using a chemical mechanical planarization method, the end of the optical fiber and the ferrule.

13. The method of claim 11, wherein the melted material creates a seal between the optical fiber and the ferrule.

14. The method of claim 11, wherein the ferrule comprises a cone-shaped portion coupled to an internal channel of the ferrule and the cone-shaped portion is occupied by a melted portion of the optical fiber.
